(19) United States
(12) Patent Application Publication
(10) Pub. No.: US 2004/0234456 A1
(43) Pub. Date: Nov. 25, 2004

(54) Device for oral administration of teething drug, other drugs and other oral administration

(76) Inventor: Gymama Slaughter, Midlothian, VA (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/843,452

(22) Filed: May 12, 2004

Related U.S. Application Data
(60) Provisional application No. 60/469,426, filed on May 12, 2003.

(51) Int. Cl. A61L 9/04; A61K 9/68; A61M 11/00

(52) U.S. Cl. 424/45; 128/200.23

(57) ABSTRACT

An oral dispensing system is provided. A device, pre-loaded with a drug, is insertible into a mouth of a patient or user having gums. Upon action of the gums with the device, the pre-loaded drug is dispensed. In an embodiment with an internal reservoir, the drug is released through holes and delivered into the mouth. In a ribbed embodiment without an internal reservoir and without holes, the drug resides on the exterior of the device and in use is delivered into the mouth.

This application claims benefit of U.S. provision application Ser. No. 60/469,426, titled “Drug administering mouthpiece and shield system,” filed May 12, 2003.

### FIELD OF THE INVENTION

The present invention is related to oral delivery. More particularly, it is a system for oral delivery especially suited for use in babies, and also having other applications.

### BACKGROUND OF THE INVENTION

There have been disclosed certain devices for oral administration of liquid formulations, such as liquid-dispensing pacifiers and a variety of other dispensing systems. See, e.g., Miller et al., U.S. Pat. No. 5,123,915 issued Jun. 23, 1992 (“Medicated pacifier”); Badalamenti et al., U.S. Pat. No. 5,957,954 issued Sep. 28, 1999 (“Teething gel applicator”); Badalamenti et al., U.S. Pat. No. 6,361,552 (“Teething gel applicator with cutter, and burstable ampule and method of making the same”); Demeter et al., U.S. Pat. No. 5,354,274 issued Oct. 11, 1994 (“Device for oral administration of liquids”); Wong et al., U.S. Pat. No. 5,780,058 issued Jul. 14, 1998 (“Oral delivery of discrete units”); Wong et al., U.S. Pat. No. 5,910,321 issued Jun. 8, 1999 (“Multiple flow path device for oral delivery of discrete units”); Wong et al, U.S. Pat. No. 5,985,324 issued Nov. 16, 1999 (“Flow controller configurations for an active agent delivery device”); Wong et al., U.S. Pat. No. 5,989,590 issued Nov. 23, 1999 (“Oral delivery of discrete units”); Wong et al., U.S. Pat. No. 6,096,003 issued Aug. 1, 2000 (“Closure system for an active agent delivery device”); Wong et al., U.S. Pat. No. 6,103,265 issued Aug. 15, 2000 (“Flow controller configurations for an active agent delivery device”); U.S. Pat. No. 6,106,845 issued Aug. 22, 2000 (“Oral delivery of discrete units”); U.S. Pat. No. 6,210,713 issued Apr. 3, 2001 (“Oral delivery of discrete units”); U.S. Pat. No. 6,333,050 issued Dec. 25, 2001 (“Oral delivery of discrete units”); U.S. Pat. No. 6,572,582 issued Jun. 3, 2003 (“Mixing system for an active agent delivery device”); U.S. Pat. No. 6,595,951 issued Jul. 22, 2003 (“Closure system . . . ”)

Conventionally, and often in separate contexts not involving dispensation of any medication or formulation, there have been suggested a variety of dental and medical mouth appliances, such as Singer et al., U.S. Pat. No. 5,823,193 issued Oct. 20, 1998 (“Dental appliance for alleviating snoring and protecting teeth from bruxism”); U.S. Pat. No. 6,318,371 issued Nov. 20, 2001 (“Mouth guard for medical use”).

However, despite all the work mentioned, there still remain unmet needs for simply, safely and effectively easing certain dental difficulties, discomfort and pain (such as teething pain in babies, etc.), and/or for orally administering formulations.

### SUMMARY OF THE INVENTION

For a patient or user (such as a baby, a human patient of any age, and certain animals and creatures) having a mouth with gums, the present invention exploits the pressure that the gums naturally may apply to an object with which the gums come in contact. Advantageously, in the present invention pressure from the gums of a patient or user may be applied to an article inserted in the mouth to dispense, from the device to the patient or user, a to-be-dispensed material that has been pre-loaded onto or into the device before the device is in the mouth.

The invention in one preferred embodiment provides an oral dispensing system, comprising: for a device that is insertible into a mouth of a patient or user having gums, pre-loading the device with a quantity of a to-be-dispensed material (such as a drug suitable for oral administration, a nutrient suitable for oral administration, etc., of which a teething drug is a most preferred example); insertion of the insertible pre-loaded device into the mouth of the patient or user; upon action of the patient or user's gums with the device, dispensation of the pre-loaded to-be-dispensed material from the device to the patient or user (such as, preferably, to-be-dispensed material being transfered from the device to the patient at or near an absorptive mouth surface).

In a particularly preferred embodiment, to-be-dispensed material is pre-loaded into an interior part of the device. Advantageously, there may be included a valve disposed to prevent backflow of to-be-dispensed material that has begun to leave the interior part of the device. The pre-loaded to-be-dispensed material within the device may be ejected from at least one aperture of the device (preferably multiple apertures, most preferably apertures located on a top and a bottom of the device), with the ejection brought about by force on the device by the gums of the mouth.

In another particularly preferred embodiment, to-be-dispensed material is pre-loaded onto at least one exterior surface of the device. Such as device that may receive exterior pre-loading need not have an internal reservoir and apertures, and may lack an internal reservoir and apertures.

In another preferred embodiment, the invention provides an oral dispenser device, comprising: an article insertible into a mouth of a patient or user having gums; a reservoir capable of holding a to-be-dispensed material, the reservoir being within the insertible article; at least one aperture from which to-be-dispensed material escapes upon application to the device of pressure delivered by the gums of the patient or user; and a mouthpiece corresponding to a gum line contour of the patient or the user and controlling release of the to-be-dispensed material (such as, most preferably a U-shaped mouthpiece). The mouthpiece may be integral with the insertible article or may be a separate piece mechanically cooperating with the insertible article, with a one-pieced device being most preferable.

In a preferred embodiment of the invention, an inventive oral dispenser device has been pre-loaded (at any time before insertion of the device into the mouth) with a to-be-dispensed material. Advantageously, an inventive article may be sized and shaped for insertion into a variety of mouths, such as a mouth of a human baby, a mouth of a geriatric patient, etc.

The invention in a preferred embodiment also provides an oral dispenser device, comprising: a ribbed article capable of holding a to-be-dispensed material, the ribbed article being insertible into a mouth of a patient or a user having gums, wherein, when inserted, at least one rib of the ribbed article contacts the gums. Optionally, and preferably, the inventive oral dispenser device includes a mouthpiece corresponding to a gum line contour of the patient or the user, with a most preferred example of such a mouthpiece being U-shaped. When a mouthpiece is included, optionally, but preferably, such an inventive oral dispenser device includes a mouth shield providing structural support and securing the mouthpiece. Preferably, texturing is provided on the mouth piece and/or the mouth shield. When such texturing is used, texturing advantageously provides structural support to secure a softer mouth piece.

In a further preferred embodiment, the invention provides an oral dispenser device, comprising: a mouthpiece of medical grade silicone, having thereon or therein a to-be-dispensed material (such as, e.g., a drug suitable for oral dispensation, etc., of which a teething drug is a preferred example), wherein the to-be-dispensed material will be released from the mouthpiece upon (A) application of gum pressure when placed in a mouth of a user and/or (B) contact with solvating saliva in a mouth of a user. As examples of such a mouthpiece may be mentioned a mouthpiece insertible into, e.g., a human baby's mouth, a mouth of a human adult, a mouth of an animal or creature having gums.

### SUMMARY OF FIGURES

FIG. 1 is a flow chart of an oral dispensing system according to the present invention.

FIG. 2 is a flow chart of an oral dispensing system according to the present invention, in one exemplary form, with a device with an inner reservoir.

FIG. 3 is a flow chart of an oral dispensing system according to the present invention, in another exemplary form, with a device which carries a to-be-dispensed material on at least one external surface.

FIG. 4A is a perspective view of an exemplary ring according to the invention. FIG. 4B is a cross-sectional view of the ring of FIG. 4A. FIG. 4C corresponds to section A-A of FIG. 4B, at a 2:1 scale. FIG. 4D corresponds to section B-B of FIG. 4B, at a 2:1 scale.

FIG. 5A shows a perspective view of an exemplary ring holder according to the invention. FIG. 5B is a cross-sectional top-to-bottom view of the ring holder of FIG. 5A. FIG. 5C is a top view of the ring holder of FIG. 5A. FIG. 5D is a cross-sectional side view of the ring holder of FIG. 5A.

FIG. 6A shows a perspective view of an exemplary shield according to the invention. FIG. 6B is a rotated view of FIG. 6A. FIG. 6C corresponds to FIGS. 6A and 6B. FIG. 6D is a cross-sectional view of FIG. 6C for section A-A, at a 2:1 scale. FIG. 6E is a cross-section view of FIG. 6C for section B-B, at a 2:1 scale.

FIG. 7 is a cross-section view of an exemplary valve according to the invention.

FIG. 8A is a perspective view of an exemplary mouthpiece according to the invention. FIG. 8B corresponds to FIG. 8A. FIG. 8C shows Section A-A of FIG. 8B, at a 3:1 scale. Circled detail “B” of FIG. 8C is shown in FIG. 8D, at a scale of 6:1. FIG. 8A depicts view “C” of FIG. 8C, at a 3:1 scale.

FIGS. 9A-9F show an exemplary reservoir mouthpiece according to an embodiment of the invention, with FIG. 9A being a perspective view. FIG. 9B is a rotated view of FIG. 9A. FIG. 9C is a cross-sectional view corresponding to FIGS. 9A-9B. FIG. 9D shows section A-A of FIG. 9C, at a 3:1 scale. FIG. 9E shows view “C” of FIG. 9D, at a 3:1 scale. FIG. 9F shows detail “B” of FIG. 9D, at a 6:1 scale.

FIG. 10 is an exploded view of an oral delivery device according to an exemplary ribbed embodiment of the present invention.

FIG. 11 is an assembled view of an oral delivery device according to an exemplary embodiment of the present invention, with FIG. 11 corresponding to the exploded view of FIG. 10.

FIG. 12 is an exploded view of an oral delivery device according to an exemplary reservoir embodiment of the present invention.

FIG. 13 is an assembled view of an oral delivery device according to an exemplary embodiment of the present invention, with FIG. 13 corresponding to the exploded view of FIG. 12.

FIG. 14A is an exemplary teether half in perspective view according to the present invention. FIG. 14B is a sectional drawing corresponding to FIG. 14A. FIG. 14C is a diagram for proportioning an inventive device with a suitable angle for insertion into a mouth.

### DETAILED DESCRIPTION OF PREFERRED EMBODIMENT

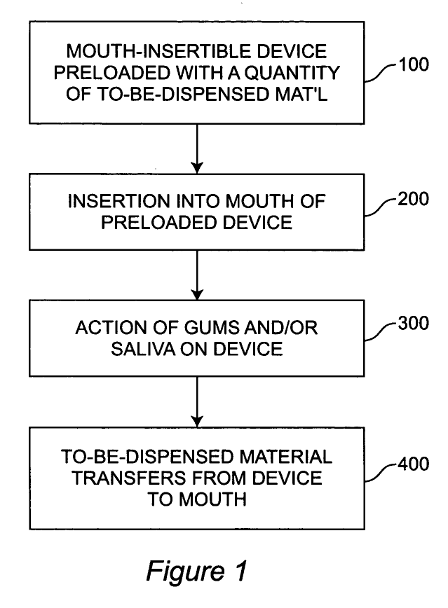

Referring to FIG. 1, in one embodiment of the invention, a device (before being placed in the mouth of the patient or user having gums) is pre-loaded 100 with a quantity of to-be-dispensed material (such as a drug or nutrient suitable for oral administration, etc.), such as pre-loading the to-be-dispensed material into the interior of the device or pre-loading the to-be-dispensed material onto an exterior surface of the device. “Pre-loading” refers to a time before the device is inserted into mouth, and pre-loading may occur, for example, during commercial manufacture, at a pharmacy, by a health care provider, by a parent or care-giver in the home, etc.

As the to-be-dispensed material may be used any material suitable for oral administration, such as a drug suitable for oral dispensation (such as a teething drug), of which a most preferred example is a drug that is suitable for oral dispensation and that is absorbed by at least one area in the mouth (such as a teething drug). Another drug besides a teething drug may be used in the invention, if the drug can be suitably absorbed via at least one mouth area. As examples of forms of the pre-loaded to-be-dispensed material may be mentioned a gel, a paste, a powder, a liquid, etc., with a gel, a paste and a powder being most preferred.

It will be appreciated that a quantity of the pre-loaded to-be-dispensed material is commensurate to accommodation by the mouth of the patient or user, with a preferred example of a quantity of the pre-loaded to-be-dispensed material being, a quantity in a range of, by volume, about 0.100 to 0.170 ml (or about 100-170 microliters) or, by mass, about 150 to 166 mg (milligrams). The invention is particularly suited for dispensing to-be-dispensed materials (such as drugs) that can be effective in small applied amounts, especially that can be formulated into an effective dosage taking into account the available geometry of an internal reservoir or an external application area that can be provided without danger or undue discomfort to a user or patient.

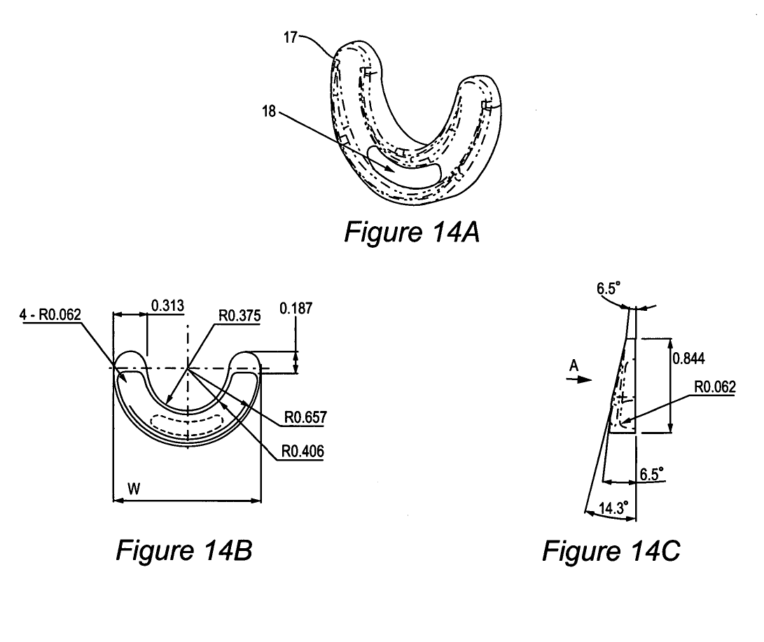

The device to be used in the present invention is not particularly limited, and there may be mentioned a device that is insertible into the mouth of a patient or user, namely, a device configured for comfort and safety of insertion into the type of mouth for which it is to be used. For example, a smaller device may be provided for babies than for adults. When providing a device for insertion into a human mouth, most preferably the device is constructed based on an angle of about 6.5 degrees for corresponding to a human jaw, with the same angle being applicable for human babies, children and adults. With regard to devices constructed for a human mouth, although the jaw angle measurement remains constant, the length dimension of the device is adjusted according to the size of the human mouth. A dimension of about 1.375 inches (total width of gum/teeth impressions) may be used for a device for inserting into a mouth of a human baby; a greater total width of gum/teeth impressions may be used for a device for inserting into a mouth of an older human child, and human adult, respectively. Desirable device dimensions may be calculated with further reference to Example 6 and FIGS. 14A-14C herein.

The material of the device is any material suitable for insertion into the mouth of a patient and for functioning as mentioned herein, with a most preferred material being a pliable plastic material, of which medical grade silicone (especially grade 6) is mentioned as a most preferred example. Latex rubber is considered less desirable to use because of being less strong.

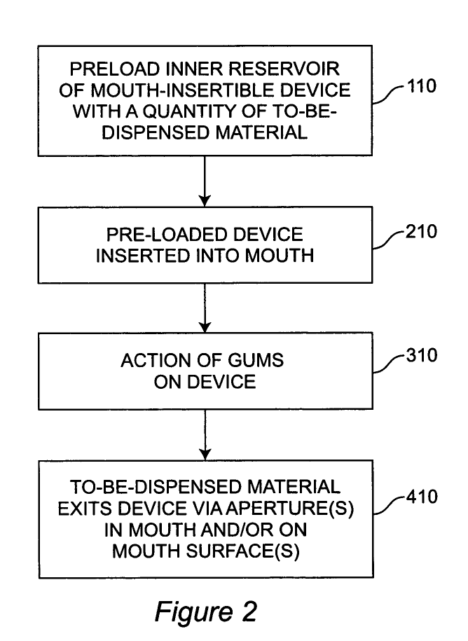

Referring again to FIG. 1, as one example of the pre-load step 100 of FIG. 1, may be mentioned a step 110 in FIG. 2, of pre-loading an inner reservoir of a mouth-insertible device with a quantity of to-be-dispensed material. As another example of the pre-load step 100 of FIG. 1, may be a mentioned a step 120 of pre-loading at least one external surface of a device with a quantity of to-be-dispensed material.

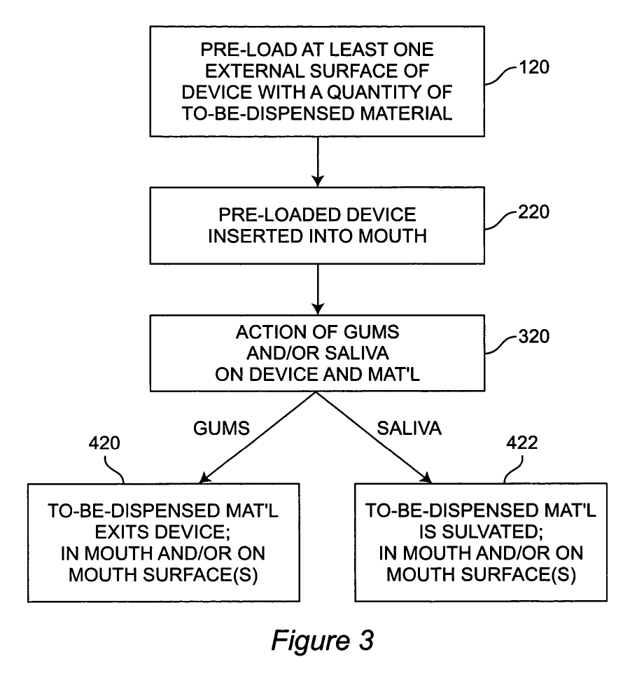

With reference to FIG. 1, according to the present invention, there is a step of insertion 200 of the preloaded device into a mouth of a patient or user, such as, for example, insertion step 210 or insertion step 220 in FIGS. 2 and 3, respectively. Advantageously, insertion 200, 210 or 220 of the article into a mouth of a patient or user is minimally invasive. Insertion 200, 210 or 220 of an article according to the invention may be by the one whose mouth it is or by someone other than the one whose mouth it is. For example, a health care professional or family member may insert the article into the mouth of a patient, or a veterinarian or pet owner may insert an article according to the invention into the mouth of an animal. The insertible article preferably is sized and shaped to minimize discomfort and/or choking hazard, and is ideally suited for a pediatric patient, a geriatric patient, an anaesthetized patient, or other patient.

Advantageously, the present invention provides a device suitable, in certain embodiments, for self administration. Preferably the device is without burrs or sharp edges or protrusions on its surface, and such absence of burrs, sharp edges and sharp protrusions is advantageous.

The insertible article according to the invention is shaped and sized so that in many uses, upon insertion, a patient naturally retains the article in his or her mouth for a desired length of time or until a desired transfer 400 has occurred. However, optionally and if needed, the article easily may be kept in the mouth of the patient for a desired length of time by relatively gentle holding-shut of the mouth with the inserted device inside the mouth.

Referring to FIG. 1, after the device is inserted 200 into the mouth of the patient or user, action 300 of at least the patient or user's gums occurs on the device. It will be appreciated that the present invention exploits normal mouth positions and/or movements and does not require or rely on a sucking action by the patient or user. In the case of a device with an internal reservoir and aperture(s), referring to FIG. 2, action 310 of the patient or user's gums applies pressure and forces the to-be-dispensed material towards and through the aperture(s). Preferably, the aperture(s) are located so that the to-be-dispensed material exits the device near an absorptive mouth surface (such as gums).

Referring to FIG. 3, in the case of an externally-loaded device, action 320 may consist of relevant action of the gums and/or saliva of the patient or user on the device. In such a case of an externally-loaded device, referring to FIG. 3, by such action 320 of the patient or user's gums and/or saliva, the pre-loaded to-be-dispensed material is transferred or dispensed 420 to the patient or user's mouth upon action of the patient or user's gums with the device and/or transferred or dispensed 422 to the patient or user's mouth upon action of saliva solvating the to-be-dispensed material. Thus, the present invention applies the action of the gums and/or saliva of a patient or user to dispense a pre-loaded to-be-dispensed material.

A volume of the pre-loaded to-be-dispensed material is selected to avoid overwhelming the patient or user, to avoid the size of the insertible device being too large to comfortably be accommodated within the mouth and to avoid too much volume or mass of a substance being released into the mouth. For example, a preferable volume for pre-loading a reservoir for use in a baby may be about 0.100 ml, with a larger amount (such as about 0.170 ml) useable in an adult. A preferable amount for pre-loading an external device for use in a baby may be about 150 mg, with a larger amount (such as about 166 mg) useable in an adult.

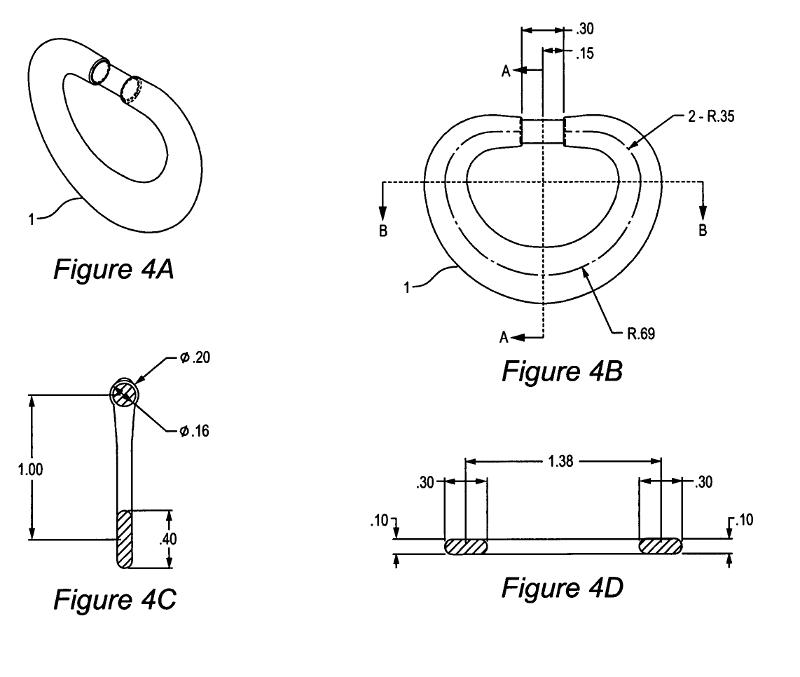

Making exemplary devices according to the invention may be appreciated with reference to FIGS. 4A-14C. Dimensions shown on the figures are for reference appreciating an exemplary embodiment, and the invention is not limited to devices of such particular dimensions.

In FIGS. 4A-4D is shown an exemplary ring 1 according to the invention, such as a teething ring. The ring 1 of FIGS. 4A-4D is sized and shaped to be received into the mouth of a patient or user.

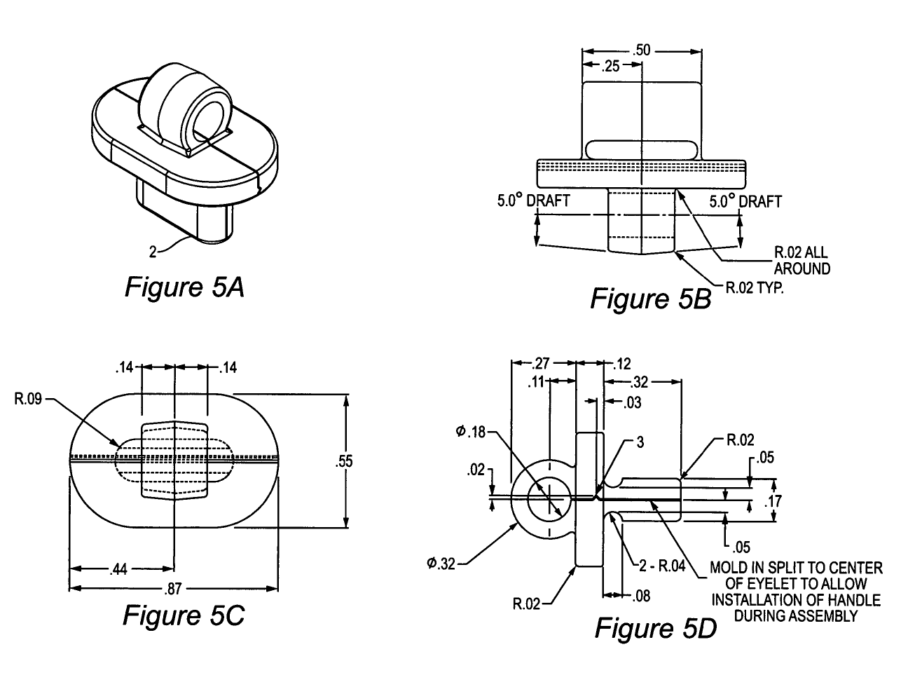

In FIGS. 5A-5D is shown an exemplary ring holder 2 according to the invention. Ring holder 2 is for holding ring 1. Preferably, ring holder 2 is formed with alignment notch 3 shown on FIG. 5D.

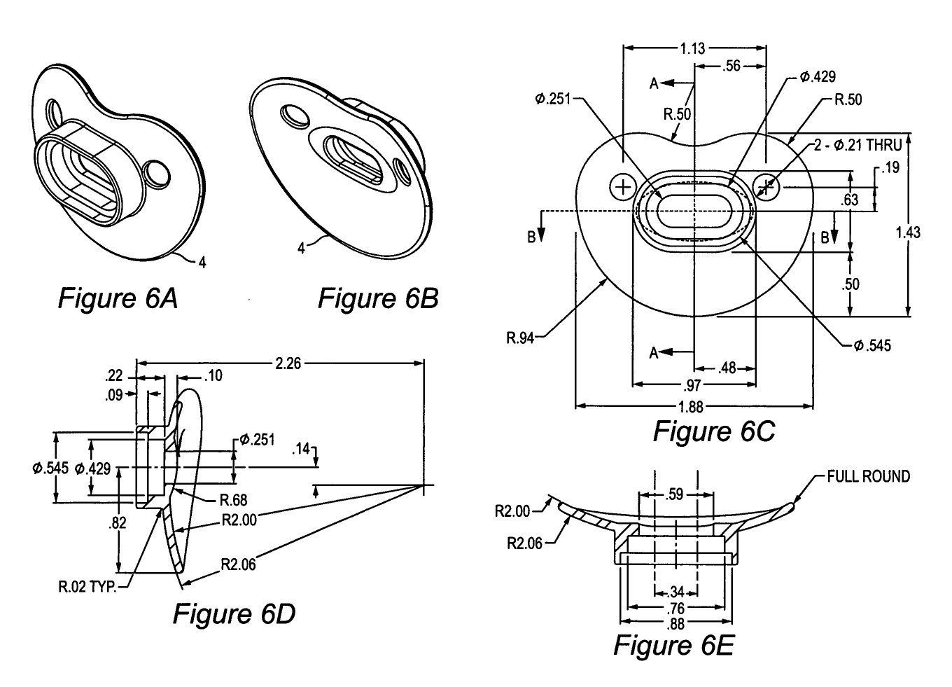

In FIGS. 6A-6E is shown an exemplary shield 4 according to the invention.

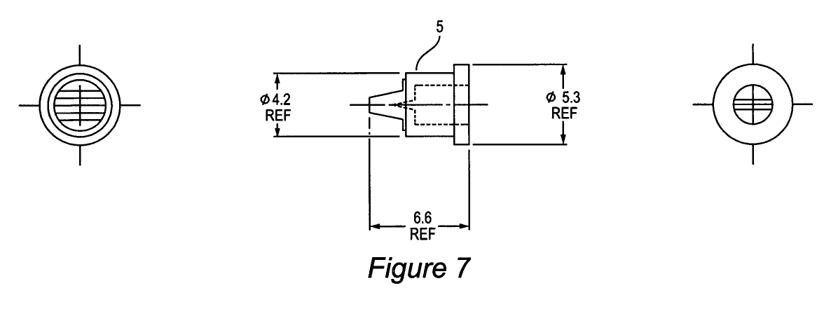

In FIG. 7 is shown a duckbill check valve 5 which is an example of a valve which may be used in the invention.

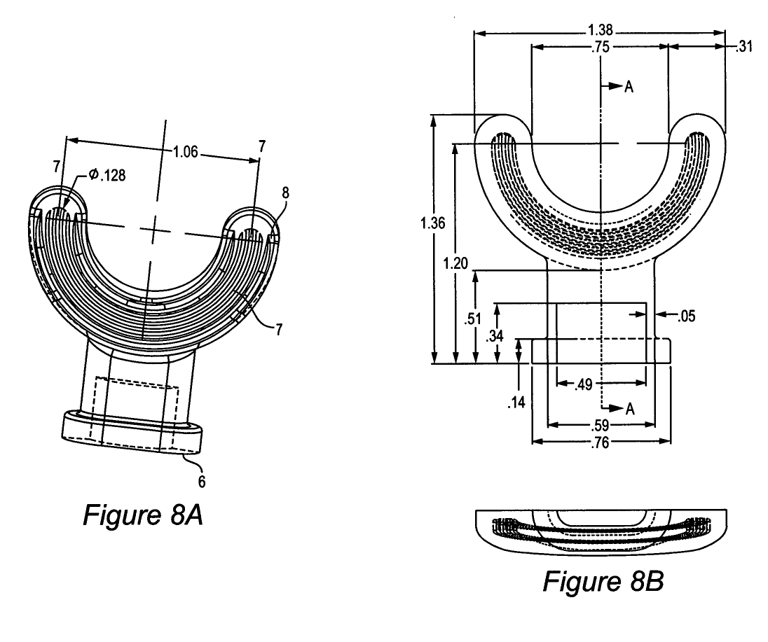

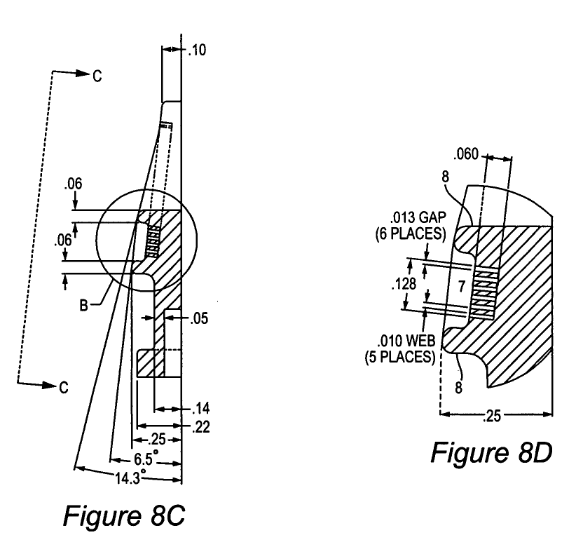

In FIGS. 8A-8D is shown a mouthpiece 6 with ribs 7. The mouthpiece 6 is U-shaped. Mouthpiece 6 includes guard 8.

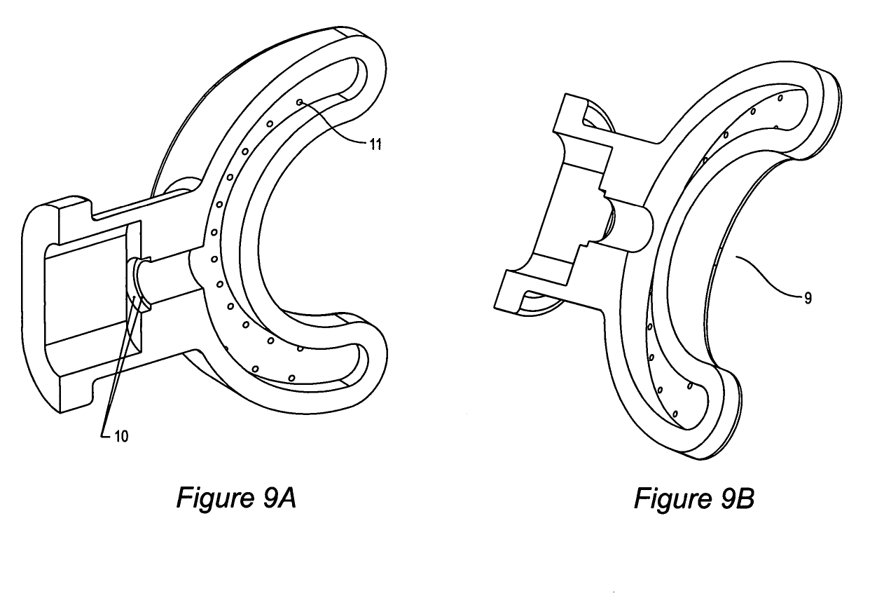

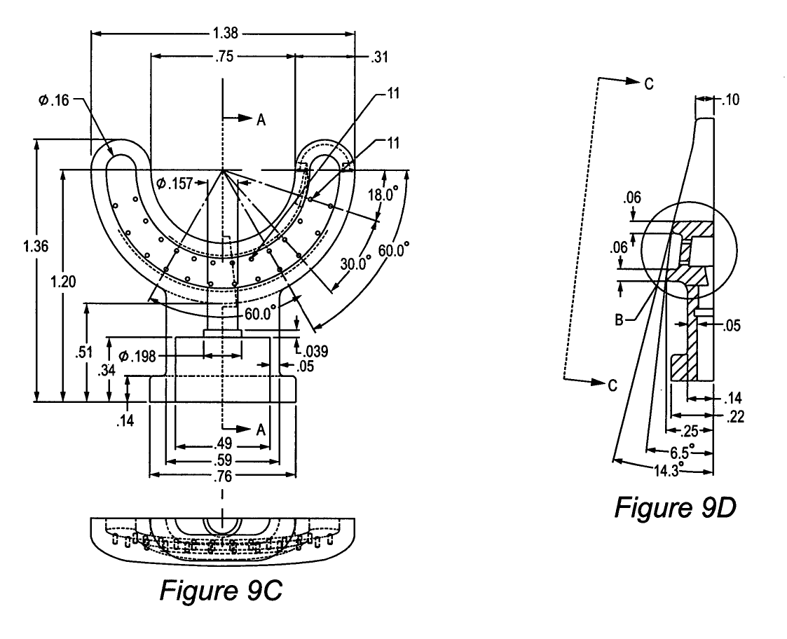

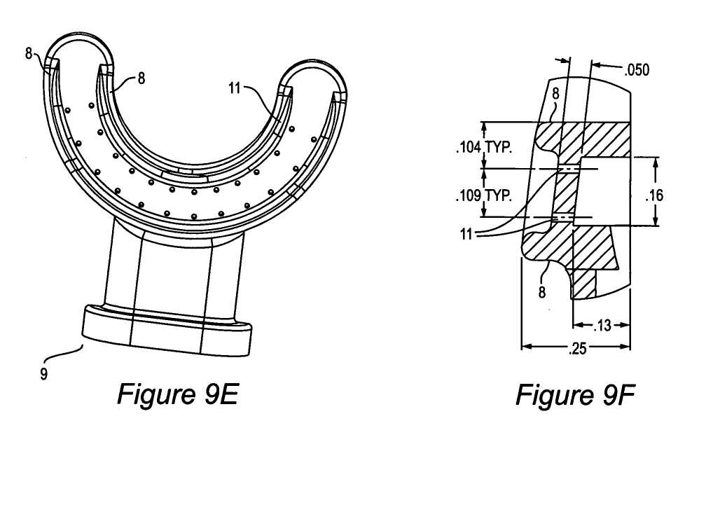

In FIGS. 9A-9F is shown a reservoir mouthpiece 9 which is U-shaped and provided with a plurality of apertures 11. For constructing a device according to the invention, at surfaces 10 (FIG. 9B) primer may be applied and then adhesive, and the mouthpiece surfaces 10 mated to surfaces of check valve 5 (FIG. 7). As an adhesive may be mentioned Henkel Loctite Medical Adhesive 5248; as a primer may be mentioned 7701.

As for apertures 11, a particularly preferred configuration of apertures is shown in FIGS. 9A-9F, without the invention being limited to such a configuration. As seen in FIG. 9C, one set of 12 apertures 11 is provided, about 0.020-inch (˜500 μM) equally spaced, at 12 degrees, and two sets of 6 holes about 0.020-inch equally spaced at 15 degrees are provided. Preferably the aperture 11 may be a nipple pore of a size in a range of about 500-900 μM (˜0.020-0.035 inch). As examples of aperture 11 may be mentioned a drug delivery capillary, a hole, etc.

With reference to the mouthpiece medicine dispenser shown in FIGS. 9C-9F, in an embodiment that is hollow and perforated, a half part is shown, and a finished part would be two halves molded together.

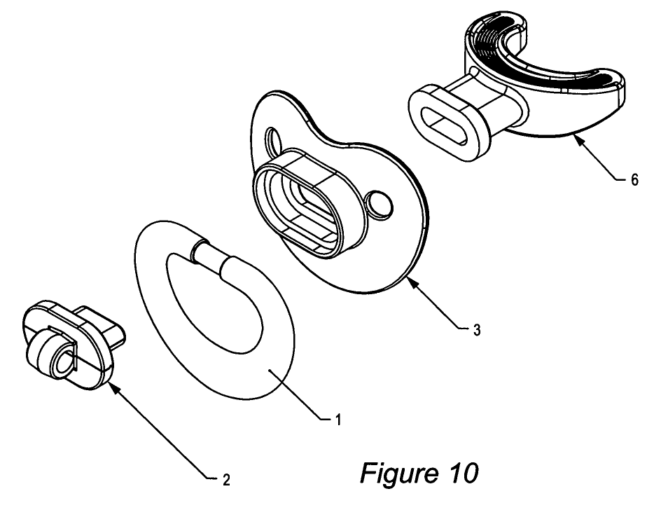

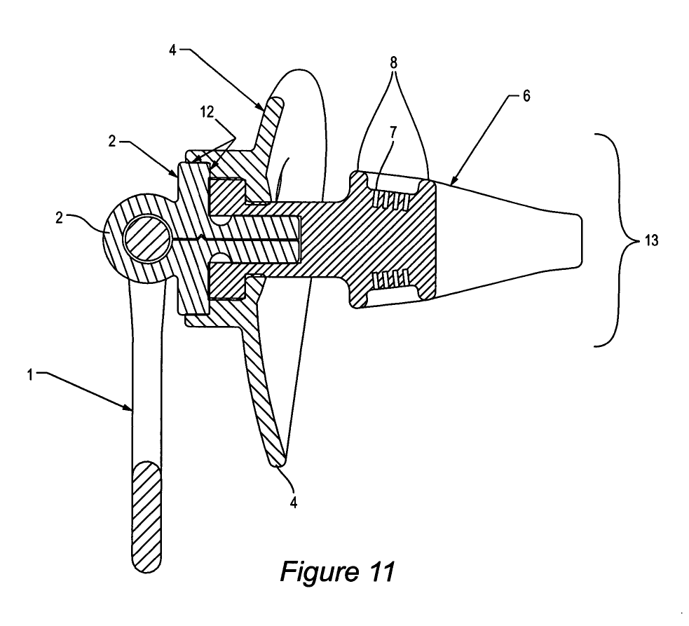

In FIGS. 10-11, assembly of an inventive oral delivery device according to a ribbed embodiment of the invention is shown, including ring 1, ring holder 2, shield 4, and mouthpiece 6. At surfaces 12 (FIG. 11), primer is applied and then adhesive at assembly. A ribbed exemplary inventive oral delivery device 13 is thus constructed.

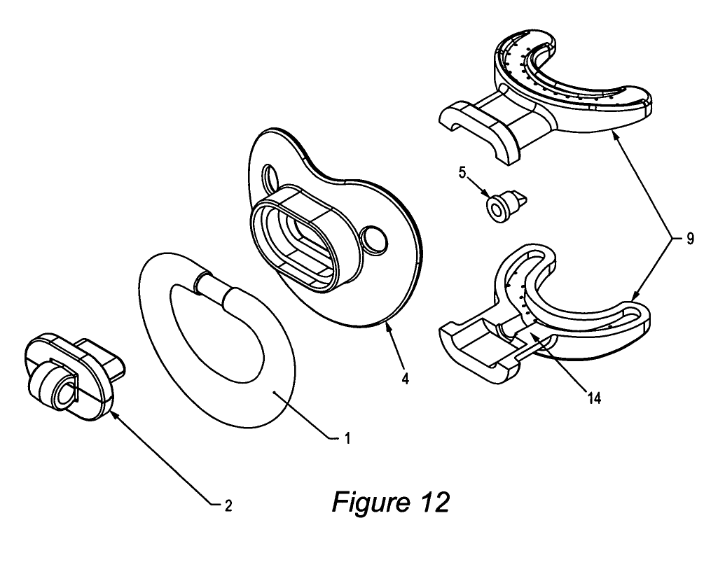

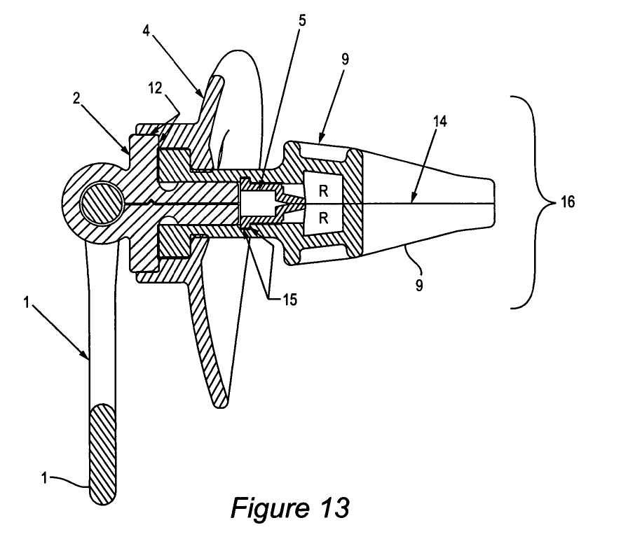

In FIGS. 12-13, assembly of an inventive oral delivery device according to a reservoir embodiment of the invention is shown, including ring 1, ring holder 2, shield 4, valve 5 and reservoir mouthpiece halves 9. In FIGS. 12-13, two identical reservoir mouthpiece halves 9 are bonded together. At surfaces 14 (FIGS. 12-13), primer then adhesive is applied to the mating surfaces of both halves. At surfaces 12 (FIGS. 12-13), bonding is carried out (as also in FIGS. 10-11). In FIG. 13, bonding also is carried out with regard to surfaces 15. In the device of FIG. 13, a reservoir R is thus provided. A exemplary inventive oral delivery reservoir device 16 is thus constructed.

For such a reservoir device 16 made according to FIGS. 12-13, in use, when the reservoir device 16 is inserted in the mouth of a patient, pressure from the gums is applied to the inserted article 16, causing to-be-administered material to exit the reservoir R and exit the device 16 by the holes 11. Upon exiting from holes 11, to-be-administered material preferably is deposited on or near an absorptive mouth surface (such as, e.g., gums). At least one valve 5 prevents backflow of the to-be-deposited material back into the device 16.

It will be appreciated that while certain configurations of ribs, holes and a reservoir are shown for illustration with regard to device 13 of FIGS. 10-11, device 16 of FIGS. 12-13 and the respective underlying figures, in the invention more generally, the quantity of each of ribs, holes and/or reservoir is not particularly limited.

Size and shape of the inventive device (such as, e.g., device 13 or 16) are not particularly limited, provided that the device is sized and shaped for insertion into a mouth of a patient, such as a human patient (who may be a baby, child, adult) or a veterinary patient having gums. Size and shape of the device are selected with regard to the patient's mouth into which the device is to be inserted, with the size and shape chosen to avoid swallowing, choking and discomfort.

Devices according to the invention may be inserted into mouths of human babies, children, adults, and certain veterinary patients. However, it will be appreciated that the dimensions of a device suitable for one patient or user may not necessarily be suitable for another patient or user.

The present invention provides a clean, safe alternative to other pain relieving mechanisms, especially pain relieving mechanisms associated with dental procedures, while being effective for dispensing medication as desired.

Devices according to the invention may be used for oral administration of a medication, such as, e.g., during teething, during medical procedures, during dental procedures, etc. The invention can be used in a variety of applications, including without limitation: a baby teether, administration of medication, dental alignment, minimizing bruxism, alleviation of snoring, minimizing pain associated with dental surgery (wisdom teeth, braces, dentures, etc.), etc.

Some examples are provided below, without the invention being limited thereto.

### EXAMPLE 1

In one preferred embodiment of the present invention, a novel teether/pacifier system is provided, wherein a drug-containing teether according to the invention is given to a baby for insertion into the mouth, and upon application by the baby of gum pressure as well as solvating saliva, the drug contained in the teether is released into the baby's mouth. Such a drug-containing teether is easy to use, relieves pain, soothes gums, is light weight, and may be suitable for over-the-counter sale.

### EXAMPLE 1A

For one example of use as a teether/pacifier, a medication (either solid (e.g. powder) or gel-like or liquid (e.g., oil)) can be applied on a disposable adhesive bristle-like section of a mouthpiece and inserted into a user's mouth. The mouth shield prevents any possibility of choking and allows for easy application, as well as quick refills. Preferably such a teether/pacifier device is textured to soothe teething pains. In addition, the device preferably is dishwasher and refrigerator safe.

### EXAMPLE 2

In one example of a dental application, there may be provided a mouthpiece having a through opening to receive medication from a reservoir into a dispensing area defining a channel to contain the medication release on both the top and bottom of the device. The drug desired to be administered can be released onto a mucoadhesive disposable bristle-like plastic, then into the mouth or onto the gum or the teeth in the case of minimizing pain associated with dental surgery (e.g., teething, teeth whitening, removal of wisdom teeth, braces, dentures, dental alignment, etc.). Upon the application of pressure (biting/chewing), the medication will be released from the reservoir through the pores on both the top and bottom dispensing area of the mouthpiece and come into contact with the patient gums.

### EXAMPLE 3

For one example of medical use, medication can be introduced through the narrower opening of the mouthpiece connecting the mouth shield. A method for oral administration of medication comprises inserting medication into the medication reservoir, with the medication reservoir being in contact with a porous opening of an outer membrane of the mouth guard. The drug thus is administered over time to the user from the reservoir through the porous outer membrane. Preferably, the device includes a through opening allowing for medical instruments (such as an endoscope, etc.) to be placed in the patient's mouth.

### EXAMPLE 4

The invention provides devices that can alleviate snoring and/or protect against teeth grinding. Such an inventive device has both a hard outer mouth shield, which provides structural support that secures the softer mouth guard, which conforms to the teeth and cushions the jaw against impact. The textured hard outer membrane serves to alleviate the pain with teething and against teeth grinding. The combination of the mouth guard and mouth shield avoids a choking hazard.

### EXAMPLE 5

There is provided an inventive device that is disposable product and/or contains an adhesive refillable element.

### EXAMPLE 6

### Angle and Width of a Mouth/Bite Imprint

In FIG. 14A, a teether half 17 has a dispensing area 18. As seen in FIG. 14B, the width w of the teether half 17 corresponds to the width of the mouth into which the device is to be inserted. In the case of a human mouth, the width w is about 1.375, where the width was obtained by biting bread and measuring the imprint. The width of the mouth is approximately same from individual to individual, except the width of the mouth varies once the molars are included. An angle of 6.5° for the inventive device for insertion into a human mouth has been calculated according to FIGS. 14B-14C and accompanying views A, B-B.

Thus, it can be seen that the present invention provides an advantageous oral dispensing system. For example, an inventive device, pre-loaded with a drug, is easily insertible, with relative comfort, into a mouth of a patient or user having gums. Upon action of the gums with the inventive device, the pre-loaded drug is dispensed. In an embodiment with an internal reservoir, the drug is released through holes and delivered into the mouth. In a ribbed embodiment without an internal reservoir and without holes, the drug resides on the exterior of the device and in use is delivered into the mouth.

It will be appreciated that variations and modifications from the embodiments set forth above may be made without departing from the spirit of the invention, and that such modifications are within the present invention.

What I claim as my invention is:

1. An oral dispensing system, comprising:
for a device that is insertible into a mouth of a patient or user having gums, pre-loading the device with a quantity of a to-be-dispensed material;
insertion of the insertible pre-loaded device into the mouth of the patient or user;
upon action of the patient or user's gums with the device, dispensation of the pre-loaded to-be-dispensed material from the device to the patient or user.

2. The oral dispensing system of claim 1, wherein the pre-loaded to-be-dispensed material is a drug suitable for oral administration or a nutrient suitable for oral administration.

3. The oral dispensing system of claim 1, wherein the to-be-dispensed material is pre-loaded into an interior part of the device.

4. The oral dispensing system of claim 3, including a valve disposed to prevent backflow of to-be-dispensed material that has begun to leave the interior part of the device.

5. The oral dispensing system of claim 1, wherein the to-be-dispensed material is pre-loaded onto at least one exterior surface of the device.

6. The oral dispensing system of claim 3, wherein the pre-loaded to-be-dispensed material within the device is ejected from at least one aperture of the device, with the ejection brought about by force on the device by the gums of the mouth.

7. The oral dispensing system of claim 6, wherein the to-be-dispensed material exits from at least one aperture in a top of the device and at least one aperture in a bottom of the device.

8. The oral dispensing system of claim 1, wherein to-be-dispensed material transfers from the device to the patient at or near an absorptive mouth surface.

9. The oral dispensing system of claim 1, wherein a quantity of the pre-loaded to-be-dispensed material is commensurate to accommodation by the mouth of the patient or user.

10. An oral dispenser device, comprising:
an article insertible into a mouth of a patient or user having gums;
a reservoir capable of holding a to-be-dispensed material, the reservoir being within the insertible article;
at least one aperture from which to-be-dispensed material escapes upon application to the device of pressure delivered by the gums of the patient or user; and
a mouthpiece corresponding to a gum line contour of the patient or the user and controlling release of the to-be-dispensed material, wherein the mouthpiece is integral with the insertible article or is a separate piece mechanically cooperating with the insertible article.

11. The oral dispenser device of claim 10, wherein the device is one-piece.

12. The oral dispenser device of claim 10, wherein the mouthpiece is U-shaped.

13. The oral dispenser device of claim 10, wherein the device is angled at about 6.5 degrees for corresponding to a human jaw.

14. The oral dispenser device of claim 10, wherein the to-be-dispensed material is selected from the group consisting of a liquid, a gel and a paste.

15. The oral dispenser device of claim 10, wherein the to-be-dispensed material includes a drug suitable for oral dispensation.

16. The oral dispenser device of claim 10, wherein the article is sized and shaped for insertion into a mouth of a human baby.

17. The oral dispenser device of claim 10, wherein the device is pre-loaded with a to-be-dispensed material.

18. The oral dispenser device of claim 10, including a plurality of apertures.

19. An oral dispenser device, comprising:
a ribbed article capable of holding a to-be-dispensed material, the ribbed article being insertible into a mouth of a patient or a user having gums, wherein, when inserted, at least one rib of the ribbed article contacts the gums.

20. The oral dispenser device of claim 19, further including a mouthpiece corresponding to a gum line contour of the patient or the user.

21. The oral dispenser device of claim 20, wherein the mouthpiece is U-shaped.

22. The oral dispenser device of claim 20, wherein the device further includes a mouth shield providing structural support and securing the mouthpiece.

23. The oral dispenser device of claim 19, wherein the to-be-dispensed material is selected from the group consisting of a liquid, a gel and a paste.

24. The oral dispenser device of claim 19, wherein the to-be-dispensed material includes a drug suitable for oral dispensation.

25. The oral dispenser device of claim 19, wherein the ribbed article is sized and shaped corresponding to insertability into a mouth of a human baby.

26. An oral dispenser device, comprising:
a mouthpiece of medical grade silicone, having thereon or therein a to-be-dispensed material, wherein the to-be-dispensed material will be released from the mouthpiece upon (A) application of gum pressure when placed in a mouth of a user and/or (B) contact with solvating saliva in a mouth of a user.

27. The oral dispenser device of claim 26, wherein the to-be-dispensed material is a drug suitable for oral dispensation.

28. The oral dispenser device of claim 27, wherein the drug is a teething drug.

29. The oral dispenser device of claim 26, the mouthpiece insertible into one selected from the group consisting of a human baby's mouth, a mouth of a human adult, a mouth of an animal or creature having gums.
